(19) United States
(12) Patent Application Publication
(10) Pub. No.: US 2009/0209911 A1
(43) Pub. Date: Aug. 20, 2009

(54) APPARATUS AND METHOD FOR PORTABLE LIQUID DRUG DELIVERY

(75) Inventors: Eugen Cabus, Eden Prairie, MN (US); Ronald Louis Bardell, Minneapolis, MN (US); Tzu-Yu Wang, Maple Grove, MN (US); Jay Gordon Schwichtenberg, New Hope, MN (US)

Correspondence Address: HONEYWELL INTERNATIONAL INC.;PATENT SERVICES, 101 COLUMBIA ROAD, P O BOX 2245, MORRISTOWN, NJ, 07962-2245, US

(73) Assignee: Honeywell International Inc., Morristown, NJ (US)

(21) Appl. No.: 12/031,033

(22) Filed: Feb. 14, 2008

(51) Int. Cl. A61M 5/155

(52) U.S. Cl. 604/140

(57) ABSTRACT

An apparatus includes a cartridge having a liquid drug reservoir configured to hold a liquid drug and a volatile liquid reservoir configured to hold a volatile liquid. The cartridge is configured so that vaporization of at least some of the volatile liquid creates a flow of the liquid drug out of the liquid drug reservoir. The apparatus also includes a flow sensor configured to monitor the flow of the liquid drug, a liquid valve configured to regulate the flow of the liquid drug, and an insertion unit configured to deliver the liquid drug to a person. The cartridge may also include a vaporized gas chamber configured to hold the gas. The gas in the vaporized gas chamber expands and pushes the liquid drug out of the liquid drug reservoir.

### TECHNICAL FIELD

This disclosure relates generally to medical devices and more specifically to an apparatus and method for portable liquid drug delivery.

### BACKGROUND

Some medication needs to be delivered on a continuous basis to a patient with as little hindrance to the patient's daily life as possible. One common example is insulin delivery. For a normal non-diabetic person, the pancreas constantly delivers small amounts of insulin to the person's blood stream to keep a blood glucose concentration within a normal range. Insulin need that is independent of meal intake is called the “basal” rate and accounts for about 50% of the body's daily insulin requirement. Also, the insulin level in a person is not constant throughout the day. Instead, it follows a natural biorhythm. Basal insulin is needed particularly in the early morning hours and less so in the late afternoon. Before noon and during the night hours, only a small amount of basal insulin is needed. The other 50% of the daily insulin requirement is delivered quickly by the pancreas after meal intake to normalize the rise in the blood glucose level associated with the meal intake.

For a diabetic person, the pancreas is deficient in producing needed insulin to regulate the glucose concentration level in that person's blood stream. Some diabetic people use hypodermic needles to inject insulin when needed. Other diabetic people use external devices, such as insulin pumps, that can deliver insulin on a continuous basis.

### SUMMARY

This disclosure provides an apparatus and method for portable liquid drug delivery.

In a first embodiment, an apparatus includes a cartridge having a liquid drug reservoir configured to hold a liquid drug and a volatile liquid reservoir configured to hold a volatile liquid. The cartridge is configured so that vaporization of at least some of the volatile liquid creates a flow of the liquid drug out of the liquid drug reservoir. The apparatus also includes a flow sensor configured to monitor the flow of the liquid drug, a liquid valve configured to regulate the flow of the liquid drug, and an insertion unit configured to deliver the liquid drug to a person.

In a second embodiment, a system includes a delivery pump and a monitoring unit configured to monitor a condition of a person. The delivery pump includes a disposable cartridge having a liquid drug reservoir configured to hold a liquid drug, a volatile liquid reservoir configured to hold a volatile liquid, and a vaporized gas chamber. The vaporized gas chamber is configured to hold gas formed by vaporization of at least some of the volatile liquid so that the gas in the vaporized gas chamber creates a flow of the liquid drug from the liquid drug reservoir. The delivery pump also includes a flow sensor configured to monitor the flow of a liquid drug, a liquid valve configured to regulate the flow of the liquid drug, and an insertion unit configured to deliver the liquid drug to the person.

In a third embodiment, a method includes creating a liquid drug flow using a vaporized gas. The method also includes monitoring a flow rate of the liquid drug flow. The method further includes adjusting a valve opening of a valve for the liquid drug flow based on a difference between the monitored flow rate and a programmed rate. In addition, the method includes delivering the liquid drug flow from the valve opening to a person.

Other technical features may be readily apparent to one skilled in the art from the following figures, descriptions, and claims.

### BRIEF DESCRIPTION OF THE DRAWINGS

For a more complete understanding of this disclosure, reference is now made to the following description, taken in conjunction with the accompanying drawings, in which like reference numerals represent like parts:

FIG. 1 illustrates an example portable drug delivery system according to one embodiment of this disclosure;

FIG. 2 illustrates an example vaporized gas-driven liquid drug delivery pump in a portable drug delivery system according to one embodiment of this disclosure;

FIG. 3 illustrates an example interface unit in a portable drug delivery system according to one embodiment of this disclosure;

FIG. 4 illustrates an example graph of pressures from various vaporized gases according to one embodiment of this disclosure; and

FIG. 5 illustrates an example method for vaporized gas-driven liquid drug delivery according to one embodiment of this disclosure.

### DETAILED DESCRIPTION

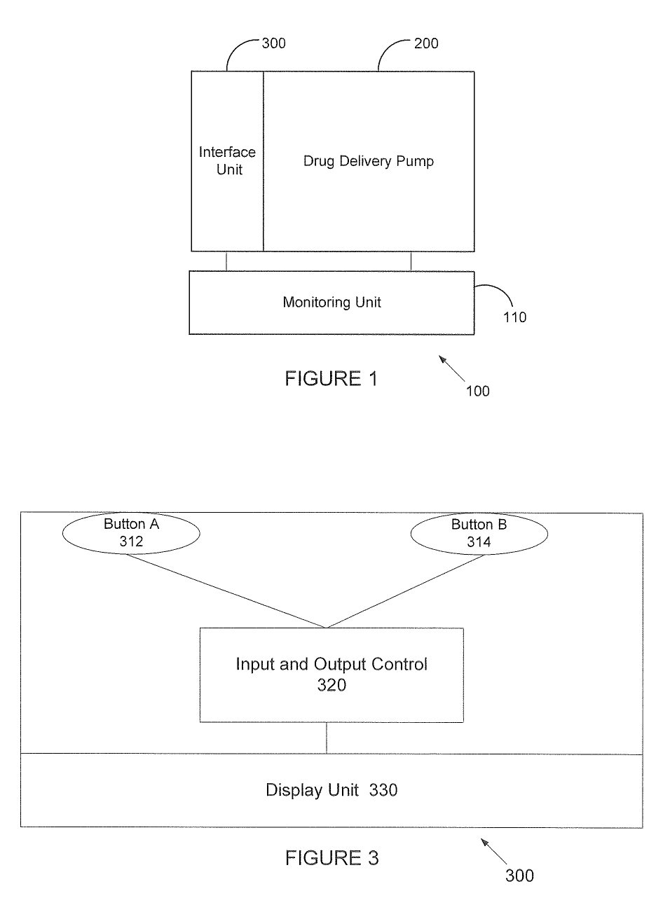

FIGS. 1 through 5, discussed below, and the various embodiments used to describe the principles of the present invention in this patent document are by way of illustration only and should not be construed in any way to limit the scope of the invention. Those skilled in the art will understand that the principles of the invention may be implemented in any type of suitably arranged device or system.

Liquid drug delivery pumps available on the market often use more expensive electric motors to deliver a liquid drug to a person. This results in both higher costs and larger or more bulky sizes, meaning the pumps are often not very comfortable for people to wear on their bodies. In addition, existing drug delivery pumps often use tubing to connect an insertion unit to the pump body. This can be cumbersome for those who are highly mobile.

FIG. 1 illustrates an example portable drug delivery system 100 according to one embodiment of this disclosure. The embodiment of the drug delivery system 100 shown in FIG. 1 is for illustration only. Other embodiments of the drug delivery system 100 could be used without departing from the scope of this disclosure.

As shown in FIG. 1, the drug delivery system 100 includes a monitoring unit 110, a drug delivery pump 200, and an interface unit 300. The monitoring unit 110 generally monitors one or more conditions associated with a person using the drug delivery system 100. For example, if the drug delivery system 100 is used to dispense insulin, the monitoring unit 110 may monitor a glucose level in the person's blood stream. In this case, the drug delivery pump 200 may increase or decrease the drug delivery rate based on a reading of the glucose level from the monitoring unit 110. In other embodiments, the monitoring unit 100 can be configured to monitor other or additional body conditions, such as blood pressure, body temperature, or pulse rate.

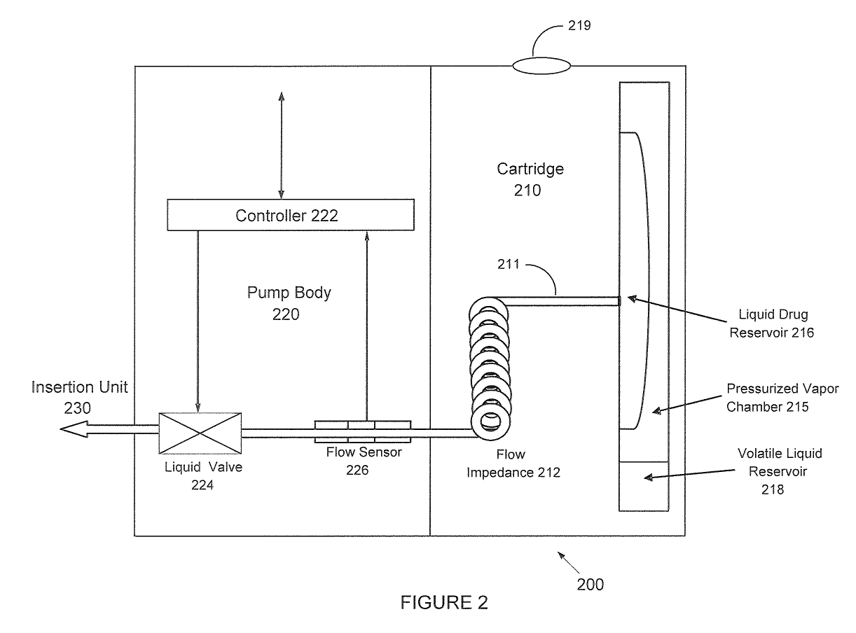

The drug delivery pump 200 provides at least one drug to the person using the drug delivery system 100. In some embodiments, the drug delivery pump 200 represents a vaporized gas-driven pump. For example, the pump 200 may include a cartridge, a pump body, and an insertion unit. The cartridge could include a volatile liquid reservoir to store a volatile liquid, which is used to produce vapor. The cartridge may also include a liquid drug reservoir to hold one or more drugs to be delivered. The pump body has a liquid valve to regulate a liquid drug flow, a sensor to monitor the liquid drug flow, and a controller to control the liquid drug flow. The insertion unit can deliver the liquid drug into the body of a person. More details of the drug delivery pump 200 are shown in FIG. 2, which is described below.

The interface unit 300 generally represents any suitable structure for receiving information from and/or providing information to a patient or other person. For example, the interface unit 300 could have an input unit and a display unit. The input unit could include input buttons, a small touch screen, or other input mechanisms. Among other things, the input unit may allow a user to program a drug delivery schedule or to initiate a drug delivery on demand. The user could input drug delivery-related information in any suitable manner, such as by entering a date, a time, an amount of liquid drug to be delivered, and a delivery action. More details of the interface unit 300 are shown in FIG. 3, which is described below.

In this example, the monitoring unit 110 can be coupled to or communicate with the drug delivery pump 200 and the interface unit 300 in a variety of ways. For example, where the monitoring target is a blood glucose level, the monitoring unit 110 may be connected to the insertion unit of the delivery pump 200 to draw a blood sample from the person using the drug delivery system 100. As another example, the monitoring unit 110 may be coupled to the delivery pump 200 via a wireless connection or an electronic wire to pass collected data (such as a blood pressure or pulse rate reading) from the monitoring unit 110 to the delivery pump 200.

Although FIG. 1 illustrates one example of a portable drug delivery system 100, various changes may be made to FIG. 1. For example, the functional division shown in FIG. 1 is for illustration only. Various components in FIG. 1 could be combined, further subdivided, or omitted and additional components could be added according to particular needs.

FIG. 2 illustrates an example vaporized gas-driven liquid drug delivery pump 200 in a portable drug delivery system according to one embodiment of this disclosure. The embodiment of the vaporized gas-driven liquid drug delivery pump 200 shown in FIG. 2 is for illustration only. Other embodiments of the delivery pump 200 could be used without departing from the scope of this disclosure.

As shown in FIG. 2, the vaporized gas-driven liquid drug delivery pump 200 includes a cartridge 210, a pump body 220, and an insertion unit 230. In this example, the cartridge 210 includes a flow impedance 212, a pressurized vapor chamber 215, a liquid drug reservoir 216, and a volatile liquid reservoir 218, and a refill opening 219 for the volatile liquid. In some embodiments, the cartridge 210 can be disposable and can be coupled to and detached from the rest of the delivery pump 200. In some embodiments, the cartridge 210 can include only the liquid drug reservoir that can be disposable, refillable or both, and the vapor chamber can be on the pump body.

The volatile liquid reservoir 218 holds a volatile liquid that can vaporize into gas under a higher temperature (such as a human body temperature). Any suitable volatile liquid could be used here, such as butane, dichloroluoromethane, ethleneoxide, ethylchloride, ethylamine, acetaldehyde, and isopentane. The choice of a particular volatile liquid could depend, among other factors, on a target pressure of the vaporized gas and an expected temperature for vaporization. The volatile liquid can be filled, refilled, or emptied for cleaning via the refill opening 219 if the cartridge is not disposable.

The pressurized vapor chamber 215 is configured to hold the vaporized gas produced from the volatile liquid stored in the volatile liquid reservoir 218. When the pressurized vapor chamber 215 is filled with vapor, the pressure increases and can thereby press or squeeze the liquid drug out of the liquid drug reservoir 216 and into a channel 211 leading into the pump body 220. In particular embodiments, the cartridge can be made in a variety of shapes so that the liquid drug delivery pump 200 can fit or attach into various positions on a person's body.

The liquid drug reservoir 216 is configured to hold at least one liquid drug to be delivered to a person using the drug delivery system 100. The liquid drug reservoir 216 includes any suitable structure for holding at least one liquid drug, such as a bag or other structure. In particular embodiments, the liquid drug reservoir 216 has a durable thin metal or metallized membrane, or other barrier to separate the liquid drug reservoir 216 from the volatile liquid reservoir 218 and the vapor chamber 215 (to prevent leakage of the vapors into the drug reservoir). The liquid drug in the liquid drug reservoir 216 may be emptied, filled, or refilled.

The channel 211 and the impedance 212 generally represents an cartridge embedded rigid structure for providing a liquid drug from the liquid drug reservoir 216 to the pump body 220. The amount of impedance from the coiled channel may depend on the amount of expected pressure from the pressurized vapor in the chamber 215. For example, if the expected vaporized pressure is high, the desired impedance may also be high. This impedance will minimize the effect of the position of the pump and of the different vibrations and movements.

The refill opening 219 can allow for refills of the volatile liquid. In some embodiments, the refill opening 219 is not present if the cartridge is disposable or the volatile liquid is on the pump body 220. Also, in some embodiment, a drug refill can be provided through the drug outlet of the cartridge.

In this example, the pump body 220 includes a controller 222, a liquid valve 224, and a flow sensor 226. The flow sensor 226 is configured to collect data (such as flow rate) about the drug flow from the cartridge 210. The flow sensor 226 is also coupled to the controller 222 so that readings about the drug flow can be reported to the controller 222. The reporting of the drug flow data can be on a scheduled basis, on demand, and/or in any other suitable manner.

The liquid valve 224 can regulate the liquid drug flow by increasing or decreasing an opening that is a normally closed (NC) valve. For example, the liquid valve could have a diaphragm, a plastic tube, and a central pole. The central pole can be slightly higher than a clamping edge of the diaphragm. Due to a pre-induced stress, the diaphragm can squeeze down on the plastic tube to close or decrease the opening of the valve. When the diaphragm is pushed up (such as by a voltage), the diaphragm moves up to open the valve more. One example of an electro-statically actuated valve is provided in U.S. Pat. No. 7,168,675, which is hereby incorporated by reference.

In some embodiments, the liquid valve 224 can be a normally closed valve using either a proportional flow control or an on-off flow control. The controller 222 may open for a short time the liquid valve 224 realizing a delivery of a small amount of the drug. The amount of liquid drug is very precisely measured, using the flow sensor signal. The controller 222 can then correctly calculate the next moment when the valve has to be opened and for how long in order to provide the right average amount of drug. In one embodiment, for delivery of insulin, this time interval between two releases can be 3-minutes long. In another embodiment, the liquid valve uses a proportional or pulse width modulation (PWM) control to regulate the flow of the liquid drug.

The controller 222 is configured to control the liquid drug flow by controlling the valve 224. For example, the controller 222 can make a control decision for the valve 224 after considering the relevant information, such as a current liquid drug flow reading from the flow sensor 226, a programmed target flow rate, and an optional input from the monitoring unit 110. The controller 222 can then send a control command to the liquid valve 224 to increase, decrease, or maintain the current flow rate. The controller 222 can also be coupled to the interface unit 300 to receive a programmed drug delivery schedule. The controller 222 can also send data to the interface unit 300 for display. The displayed data could include a current delivery schedule, a current flow rate, a current time, and a current reading from the monitoring unit 110. The controller 222 may include a memory to store the delivery schedules programmed by the user or any other information used, generated, or collected by the controller 222. In some embodiments, the controller can have a wireless communication option to send data to a local computer that eventually has a network connection to a centralized monitoring system.

In this example, the insertion unit 230 includes an insertion needle, such as a disposable micro-needle, or other disposable structure for delivering a liquid drug into a person using the drug delivery system 100. In some embodiments, the insertion unit 230 is an integral part of the drug delivery pump 200 and is directly coupled to the pump body 220 without any tubing. Without any external tubing connection, the insertion unit 230 increases the degree of portability and the ease of use of the system 100 being directly attached to the body.

Although FIG. 2 illustrates one example of a vaporized gas-driven liquid drug delivery pump 200 in a portable drug delivery system, various changes may be made to FIG. 2. For example, the various components in FIG. 2 could have any suitable size, shape, and configuration. Also, various components in FIG. 2 could be replicated any number of times according to particular needs.

FIG. 3 illustrates an example interface unit 300 in a portable drug delivery system according to one embodiment of this disclosure. The embodiment of the interface unit 300 shown in FIG. 3 is for illustration only. Other embodiments of the interface unit 300 could be used without departing from the scope of this disclosure.

In this example, the interface unit 300 includes two input buttons 312 and 314, an input and output control 320, and a display screen 330. The two input buttons 312 and 314 can allow a user to provide input data to the drug delivery system 100, such as to the controller 222 in the delivery pump 200. As a particular example, the two input buttons 312 and 314 can allow the user to add, modify, and delete a liquid drug delivery schedule. In some embodiments, a liquid drug delivery schedule may include a start time, an end time, and an amount of liquid drug to be delivered. The buttons 312 and 314 may also allow the user to activate a delivery schedule on demand to start the drug delivery immediately. The user can also change the start time, the end time, and the delivery amount through one or more of the buttons. The buttons 312 and 314 represent any suitable structures for providing input data. The use of two physical buttons is for illustration only. Any suitable number of buttons could be provided, and the buttons could be physical buttons or “soft” buttons presented on the display screen 330.

The display screen 330 can display information to the user, such as a current time, a current delivery schedule, and a current status of the liquid drug delivery pump 200. Any other information can be displayed to the user. In some embodiments, the current time, current delivery schedule, and current pump status could be displayed by default, and the user can modify the displayed data using the buttons 312 and 314. The display screen 330 can represent any suitable display, such as an LCD screen.

The input and output control 320 of the interface unit 300 can control the input and output data flow. For example, the input and output control 320 can communicate with the controller 222 of the pump body 220 to receive data to be displayed. The input and output control 320 can also send data entered by the user to the controller 222, such as data defining a delivery schedule. In some embodiments, the input and output control 320 may have a memory to store at least some of the data.

Although FIG. 3 illustrates one example of an interface unit 300 in a portable drug delivery system, various changes may be made to FIG. 3. For example, the functional layout of the interface unit 300 is for illustration only, and additional buttons or other input or output mechanisms may be used. Also, various components in FIG. 3 could be combined, subdivided, or omitted and additional components could be added according to particular needs.

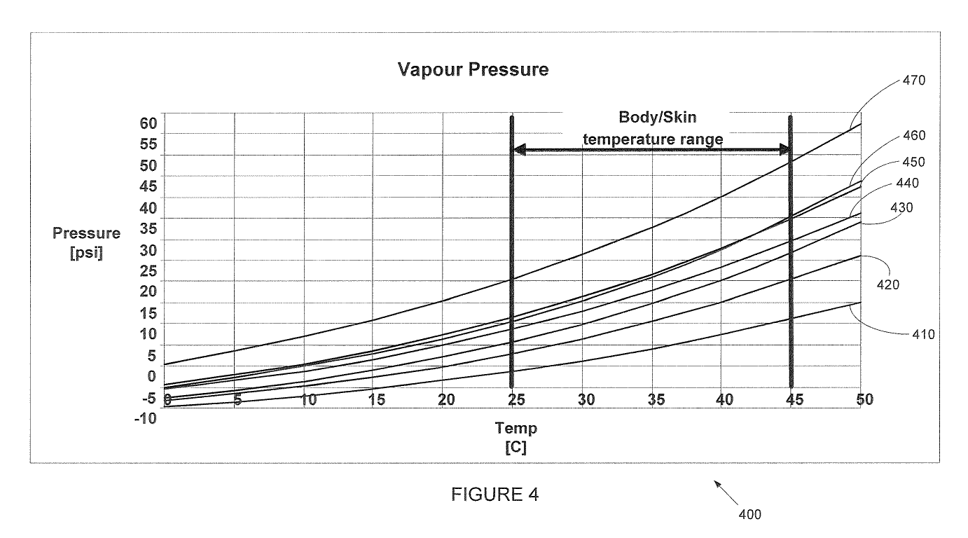

FIG. 4 illustrates an example graph 400 of pressures from various vaporized gases according to one embodiment of this disclosure. The vaporized gases associated with the graph 400 are for illustration only. Any other suitable vaporized gas could be used in the drug delivery system 100 without departing from the scope of this disclosure.

Different volatile liquids may produce different amounts of vaporized gas pressures under the same temperature. The choice of a volatile liquid for the vaporized gas-driven liquid drug delivery pump 200 can depend on the temperature of the operating environment and the target liquid flow rate (among other factors). FIG. 4 shows various vaporized gas pressures, in terms of pounds per square inch, produced from different volatile liquids under varying temperatures. Within the temperature range of a normal operating environment (such as a human body and skin temperature range of 25° C.-45° C.), the volatile liquid butane (represented by line 470) produces the highest amount of vaporized gas pressure. The volatile liquids dichloroluoromethane (represented by line 460), ethleneoxide (represented by line 450), ethylchloride (represented by line 440), ethylamine (represented by line 430), acetaldehyde (represented by line 420), and isopentane (represented by line 410) yield descending amounts of vaporized gas pressures.

Although FIG. 4 illustrates a graph 400 of example pressures from various vaporized gases, any other volatile liquid(s) can be used with the liquid drug delivery pump 200 in any suitable manner.

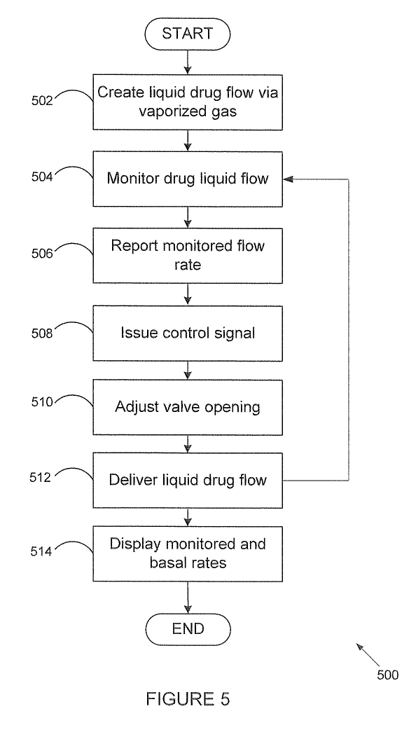

FIG. 5 illustrates an example method 500 for vaporized gas-driven liquid drug delivery according to one embodiment of this disclosure. The embodiment of the method 500 shown in FIG. 5 is for illustration only. Other embodiments of the method 500 could be used without departing from the scope of this disclosure.

A liquid drug flow is created using vaporized gas pressure and opening the NC liquid valve at step 502. For example, when the vaporized-gas driven liquid drug delivery pump 200 is attached to a patient, a rise in temperature causes the volatile liquid to vaporize and increase the vaporized gas pressure in the pressurized vapor chamber 215. In turn, this presses the liquid drug reservoir 216, causes the liquid drug to flow out of the liquid drug reservoir 216 and into the channel 211 when the NC valve is opened.

The liquid drug flow is monitored at step 504. For example, as the liquid drug flows through the flow sensor 226, a measurement can be taken at the flow sensor 226 to determine a current flow rate. Monitoring can be performed in a scheduled manner, on demand, or in any other suitable way. On-demand monitoring can produce an instantaneous reading of the liquid drug flow as instructed by the controller 222.

The monitored flow rate is reported at step 506. For example, the flow sensor 226 can report the flow rate to the controller 222. As with the monitoring itself, the reporting of the monitored flow data can be on-demand, scheduled, or performed in any other suitable way. A scheduled drug flow report may follow the same schedule as the drug flow monitoring, or it may be on a different schedule.

A control signal is issued at step 508. For example, the controller 222 can issue a control command to the valve 224. The controller 222 may consider the programmed flow rate (if there is one available), a reported current flow rate, and other information (such as the presence of an on-demand delivery command from the user). This can also include the controller 222 making a decision on an amount of adjustment to be made to the current flow rate (and for how long).

The valve opening is adjusted at step 510. This can include the controller 222 increasing or decreasing a voltage to expand or shrink a diaphragm of the liquid valve 224 to allow for a bigger or smaller opening for the liquid drug flow.

The liquid drug flow is delivered to a patient at step 512. For example, the insertion unit 230 could push the liquid drug through a disposable micro-needle and into the body of the patient. The liquid drug flow rates are displayed at step 514. For example, the interface unit 300 could receive the updated flow rate from the controller 222 so that the interface unit 300 can display the updated flow rate on the display screen 330.

Although FIG. 5 illustrates one example of a method 500 for vaporized gas-driven liquid drug delivery, various changes may be made to FIG. 5. For example, while shown as a series of steps, various steps in FIG. 5 could overlap, occur in parallel, occur in a different order, or occur multiple times. As a particular example, the drug flow rate could be monitored on a continuous basis, and the flow rate can be reported to the controller 222 and used to determine whether further adjustments are needed. In some cases, it may take multiple iterations to reach a target flow rate. Also, the method 500 has been described as involving an interface unit, a pump body, and a cartridge of a vaporized gas-driven liquid drug pump. In other embodiments, additional component, such as a monitoring unit, can also be involved.

It may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. The term “controller” means any device, system, or part thereof that controls at least one operation. A controller may be implemented in hardware, firmware, software, or some combination of at least two of the same. The functionality associated with any particular controller may be centralized or distributed, whether locally or remotely.

While this disclosure has described certain embodiments and generally associated methods, alterations and permutations of these embodiments and methods will be apparent to those skilled in the art. Accordingly, the above description of example embodiments does not define or constrain this disclosure. Other changes, substitutions, and alterations are also possible without departing from the spirit and scope of the invention, as defined by the following claims.

What is claimed is:

1. An apparatus comprising:
a cartridge comprising a liquid drug reservoir configured to hold a liquid drug and a volatile liquid reservoir configured to hold a volatile liquid, the cartridge configured so that vaporization of at least some of the volatile liquid creates a flow of the liquid drug out of the liquid drug reservoir;
a flow sensor configured to monitor the flow of the liquid drug;
a liquid valve configured to regulate the flow of the liquid drug; and
an insertion unit configured to deliver the liquid drug to a person.

2. The apparatus of claim 1, wherein the volatile liquid vaporizes into a gas and results in a gas pressure depending on a cartridge temperature.

3. The apparatus of claim 2, wherein:
the cartridge further comprises a vaporized gas chamber configured to hold the gas; and
the gas in the vaporized gas chamber expands and pushes the liquid drug out of the liquid drug reservoir.

4. The apparatus of claim 1, wherein the volatile liquid is at least one of: butane, dichloroluoromethane, ethleneoxide, ethylchloride, ethylamine, acetaldehyde, and isopentane.

5. The apparatus of claim 1, further comprising a controller configured to receive data associated with the flow of the liquid drug from the flow sensor.

6. The apparatus of claim 5, wherein the controller is further configured to send a control command to the liquid valve to regulate the flow of the liquid drug.

7. The apparatus of claim 6, wherein the controller is configured to generate the control command based on one or more of: a current reading of the flow of the liquid drug, a current time, a programmed basal rate, and an on-demand adjustment to the basal rate.

8. The apparatus of claim 1, further comprising an interface unit comprising an input and a display.

9. The apparatus of claim 8, wherein the input comprises one or more buttons that allow a user to program one or more liquid drug delivery schedules and to activate a programmed drug delivery schedule.

10. The apparatus of claim 8, wherein the display is configured to present one or more of: a current time, a programmed basal delivery rate, a reading of a current liquid drug flow rate, and a temporary adjustment to the basal delivery rate.

11. The apparatus of claim 1, wherein the liquid drug reservoir is flexible and is separated from the volatile liquid reservoir by a barrier.

12. The apparatus of claim 1, wherein the insertion unit is a disposable micro-needle coupled to a pump body without tubing and the liquid valve is a normally closed valve that uses one of: a proportional flow control and an on-off flow control.

13. The apparatus of claim 1, wherein the liquid drug comprises insulin.

14. A system comprising:
a delivery pump comprising:
a disposable cartridge comprising a liquid drug reservoir configured to hold a liquid drug, a volatile liquid reservoir configured to hold a volatile liquid, and a vaporized gas chamber, the vaporized gas chamber configured to hold gas formed by vaporization of at least some of the volatile liquid so that the gas in the vaporized gas chamber creates a flow of the liquid drug from the liquid drug reservoir;
a flow sensor configured to monitor the flow of a liquid drug;
a liquid valve configured to regulate the flow of the liquid drug; and
an insertion unit configured to deliver the liquid drug to a person; and
a monitoring unit configured to monitor a condition of the person.

15. The system of claim 14, wherein the delivery pump further comprises:
a controller configured to issue control commands to the liquid valve based on data received from the flow sensor; and
an interface unit configured to display data.

16. The apparatus of claim 15, wherein the condition of the person comprises at least one of: a glucose level, a blood pressure, a body temperature, and a pulse rate.

17. The system of claim 14, wherein the delivery pump is configured to be placed at different positions of the person.

18. A method comprising:
creating a liquid drug flow using a vaporized gas;
monitoring a flow rate of the liquid drug flow;
adjusting a valve opening of a valve for the liquid drug flow based on a difference between the monitored flow rate and a programmed rate; and
delivering the liquid drug flow from the valve opening to a person.

19. The method of claim 18, further comprising:
identifying a second monitored flow rate of the liquid drug flow; and
readjusting the valve opening based on the second monitored flow rate.

20. The method of claim 19, further comprising displaying a start time, an end time, a basal rate, and the second monitored flow rate.
